(19) United States
(12) Patent Application Publication
(10) Pub. No.: US 2012/0136339 A1
(43) Pub. Date: May 31, 2012

(54) PROTECTIVE SLEEVE FOR A CATHETER

(76) Inventor: Peter Schlumpf, Ellikon an der Thur (CH)

(21) Appl. No.: 13/389,001

(22) PCT Filed: Jul. 14, 2010
(86) PCT No.: PCT/EP2010/060152

§ 371 Date: Feb. 6, 2012

(30) Foreign Application Priority Data
Aug. 7, 2009 (CH) 01231/2009

(51) Int. Cl. A61M 25/00

(52) U.S. Cl. 604/523

(57) ABSTRACT

The invention relates to a protective sleeve (1) in the form of a hose (4), having an outer lumen (40) and an inner lumen (42) disposed eccentrically therein. The outer lumen (40) is delimited to the exterior by an outer wall (41), and the inner lumen (42) is delimited by an inner wall (43). The two walls (41, 43) transition into a common wall segment (44) which has a thinnest region (48) acting as a membrane. Pressure sensor windows (11) can be provided in said region. Said pressure sensor windows are closed by a highly flexible, thin film. This can be done by means of a shrink film tube. A reusable pressure sensor catheter can be inserted into the inner lumen (42), while a rinsing solution can be introduced into the patient through the outer lumen. Reusable pressure sensor catheters can be used a plurality of times without requiring a sterilization process. The protective sleeve (1) is replaced after each use.

The present invention relates to a protective sleeve for a catheter for performing intracorporeal measurements. Catheters are in principle tubes or hoses with which hollow organs such as the urinary bladder, stomach, intestine, vessels, etc., but also the ear and heart can be probed, emptied, filled or rinsed. This is done for diagnostic or therapeutic reasons.

Sleeves which are passed over such catheters are known per se. In most cases, such sleeves or sheathing is used to improve the guidance of highly flexible catheters. Such sheathing is preferably known by the term “sleeve” in medical technology, as described in the document WO2009/043026, for example. The sleeve shown there allows a balloon catheter to be guided by shaping the sleeve according to a nasal passage and thereby allowing improved guidance of the balloon catheter.

However, relatively short protective sleeves, which are simply inverted over the balloon area of a balloon catheter, are also known. Reference is made to the document US2008-009799, for example, in this regard. Protective sleeves for storing catheters are also known. Such a protective sleeve is disclosed in Japanese Patent Application JP 2001025473, for example. Although the protective sleeves for catheters are usually made of a flexible plastic, German Utility Model DE 29807045U, for example, describes such a protective sleeve comprised only of link elements.

Finally, the document WO 01/02045 discloses a universal protective sleeve for catheters. This protective sleeve is used to cover the extracorporeal portion of a catheter to thereby largely prevent infections.

Most of the approaches disclosed here serve only therapeutic purposes. Protective sleeves and catheters which serve diagnostic purposes are not known from this standpoint.

Catheters with pressure sensors are known for diagnostic purposes; they are used in urology and gastrology in particular. Constrictions, abnormalities or the closing force of so-called sphincter muscles are to be measured by means of such catheters with pressure sensors. Until a few years ago, these measurements were performed by means of catheters, which could be reused repeatedly. However, disposable catheters from the company Clinical Innovations, Inc. (USA) have become increasingly popular since the labor and time required for cleaning and sterilization were too great for many physicians and clinics. These disposable catheters are comprised of a multi-luminal tube, wherein a central lumen is present for passing liquids through, and two lumens running practically in the wall of the main lumen so that compressed air can be passed through them into two ring-shaped chambers arranged distally. As soon as these ring chambers are passed through a constriction, a pressure is exerted on these ring chambers and this pressure is then measured extracorporeally. Since the actual pressure probes are situated at a great distance from the measurement site, these measurements are of course inaccurate. However, since the catheter is itself an inexpensive plastic part, the catheter may be used as a disposable catheter.

Reusable catheters consist of a plastic tube in which multiple pressure sensors are arranged, distributed over the length, and whose electrical lines are passed outward centrally through the tube. The entire reusable catheter must consequently be cleaned and sterilized after each use. In contrast with the common opinion that this is expensive because of the labor and/or time associated with it, this opinion is not correct. However, it is a fact that with the highly sensitive catheters having thin conductors leading to the electrical pressure sensors the pressure sensors are destroyed due to incorrect handling during the sterilization process.

For this reason, the applicant has accepted the object of creating a protective sleeve for a catheter for performing intracorporeal measurements.

This object is achieved by a protective sleeve having the features of Patent claim 1. Additional advantageous embodiments are derived from the dependent patent claims.

A preferred embodiment is depicted in the accompanying drawings and is explained in detail on the basis of the following description.

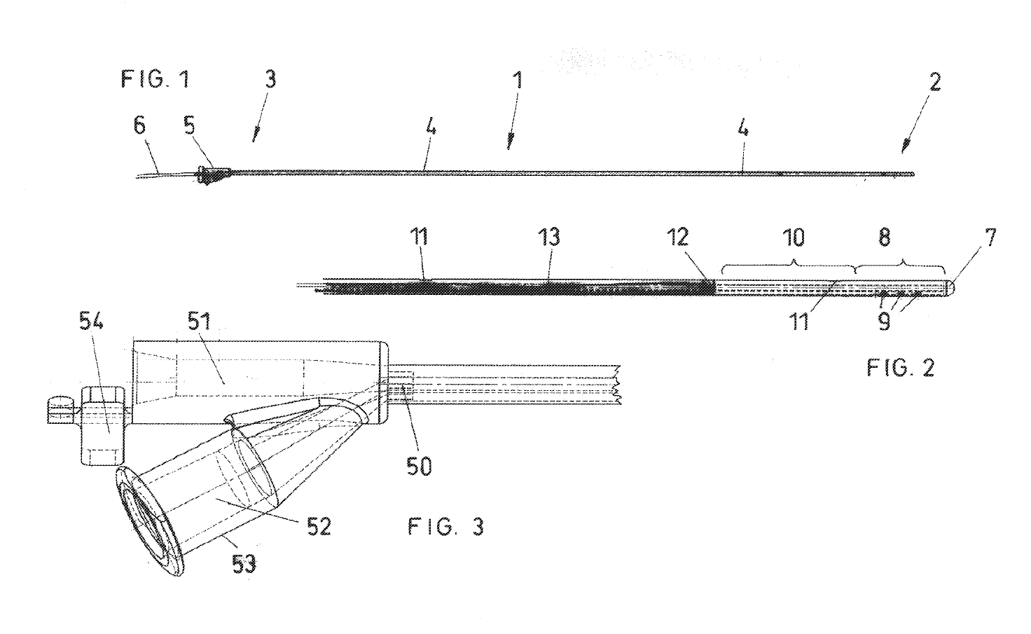

FIG. 1 shows the protective sleeve according to the invention with the insertion connection connected as a whole, and

FIG. 2 shows the proximal end of the protective sleeve on a large scale, while

FIG. 3 shows the distal end with the insertion connection on a larger scale.

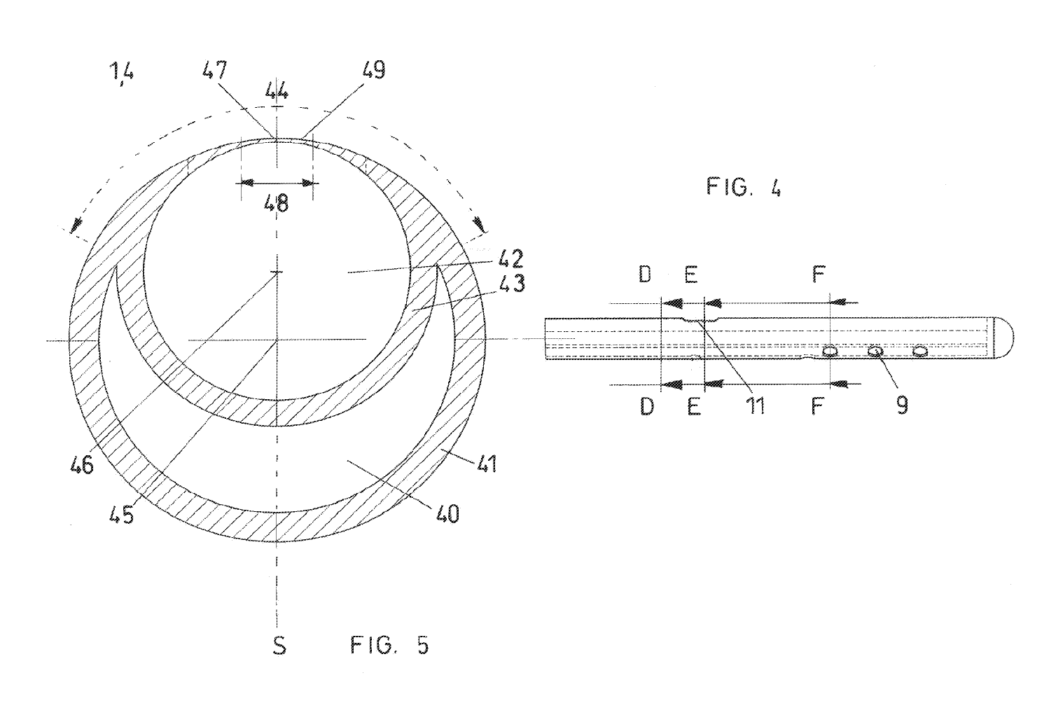

FIG. 4 in turn shows the proximal end of the protective sleeve with various lines of sectional lines.

FIG. 5 shows the diametric section along the line D-D in FIG. 4, enlarged, and

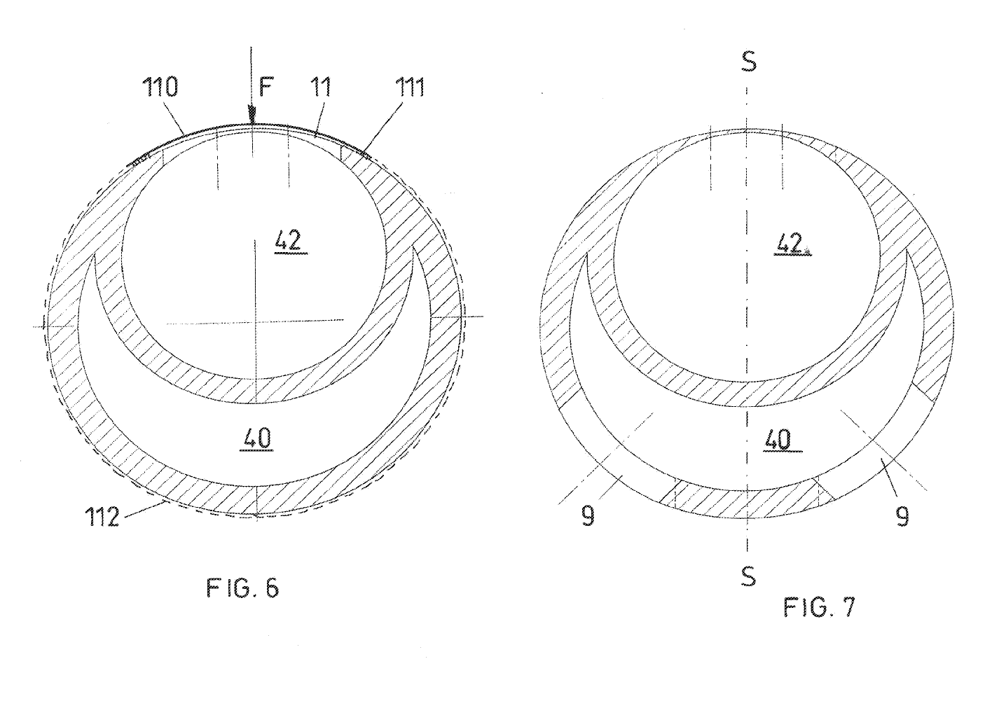

FIG. 6 shows the section along line E-E, and

FIG. 7 shows the diametric section along line F-F.

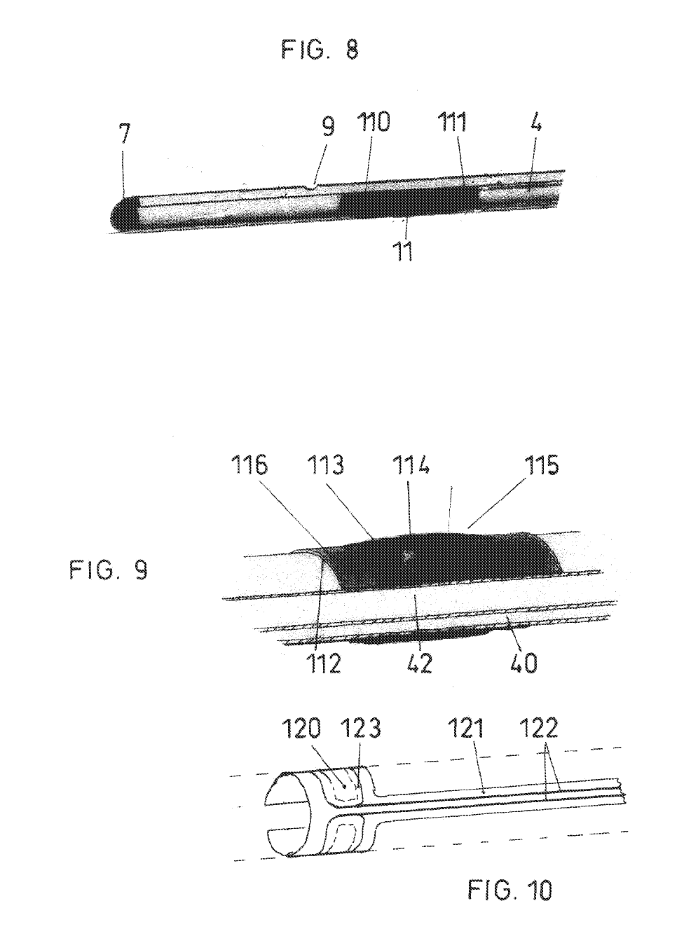

FIG. 8 shows a proximal end area of the protective sleeve with a cover over the window in the area of a pressure sensor for a unidirectional measurement, and

FIG. 9 shows the same view for a multidirectional measurement.

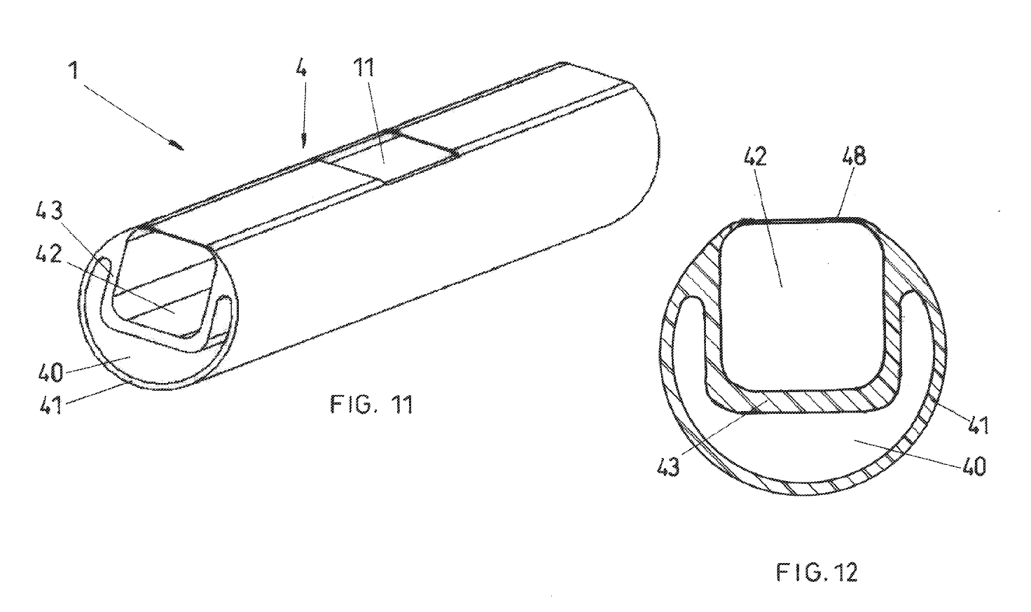

FIG. 10 shows a resistance-measuring probe for mounting on the protective sleeve according to the invention.

FIG. 11 shows an especially preferred embodiment of the protective sleeve in a perspective diagram, and

FIG. 12 shows this protective sleeve according to FIG. 11 in a sectional view.

The protective sleeve according to the invention is labeled as 1 on the whole. The protective sleeve 1 has a proximal end 2 and a distal end 3. The total length of the protective sleeve 1 is formed by a dual-lumen tube 4. The tip, i.e., the proximal end of the protective sleeve 2 is usually closed. The distal end 3 of the protective sleeve 1 is provided with an insertion connection 5. The corresponding reusable catheter 6 can be inserted through the insertion connection 5. The catheter 6 usually has a plurality of pressure sensors as well as the corresponding power supply lines and signal lines. Since the actual catheter is not a direct part of the invention, it will not be discussed further here. The proximal end 2 of the protective sleeve 1 is shown on an enlarged scale in FIG. 2. It can be seen that the protective sleeve 1 is closed and sealed by means of a tube closure 7. This tube closure 7 may be an attachable plastic part, which is welded or glued to the hose 4 for the sake of safety, or the tube closure 7 may also be closed simply by pressing the lumen together and then welding it.

In many applications of catheters for performing intracorporeal measurements, liquids must be supplied to perform rinsing operations, on the one hand, and to fill a bladder up to an emptying pressure, for example, on the other hand. In this regard, a rinse opening area 8 is then provided on the tube closure 7. A plurality of rinse openings 9 are provided in this rinse opening area 8. These rinse openings 9 communicate only with the exterior of the two lumens. A pressure-measuring area 10 then follows at the rinse opening area 8. One or more pressure sensors are arranged in this pressure-measuring area 10, which extends over a shorter or longer distance, depending on the application. These pressure sensors may in principle be arranged directly on an outer shared wall segment which is designed to be so thin that this wall segment forms a membrane. However, depending on the intended purpose, the protective sleeve 1 must be made of a relatively strong plastic, so that then this outer shared wall segment nevertheless does not have a great enough sensitivity to pressure, even if it is designed to be very thin and thus indeed forms a membrane. In this case, several pressure sensor windows 11 are provided in the pressure-measuring area 10. This will be discussed in greater detail below. In addition, an electrical resistance sensor 12 with corresponding printed conductors 13 may also be applied to the outside of the protective sleeve 1. This will also be discussed in greater detail below.

FIG. 3 shows the insertion connection 5 placed on the distal end of the protective sleeve 1. For this purpose, the tube 4 is connected by means of a plug-in nipple 50 adapted to the dual-lumen tube 4. The nipple 50 is thus part of the insertion connection 5 which is made of plastic on the whole. The insertion connection 5 has an insertion opening 51, which tapers conically in one or more stages. The measuring catheter, which is to be reused repeatedly, is inserted through this insertion opening 51. This conical insertion opening 51 thus facilitates the insertion of the reusable catheter. In addition to this insertion opening 51 in the insertion connection 5, an inlet line 52 of a lateral opening 53 opens on the insertion connection 5. In addition, an operating lever 54 with which the inserted catheter probe can be rotated in the protective sleeve 1 to align the sensor(s), so that it goes/they go in front of the pressure sensor window(s) 11, can be seen on the insertion connection 5. The shape and embodiment of the insertion connection 5 described here of course represent only a preferred embodiment, which is based essentially on the known insertion connections, but of course other embodiments may also be used. It is essential, however, for the nipple 50 of the protective sleeve 1 to be designed accordingly.

The actual protective sleeve is described in greater detail below. FIG. 4 shows the proximal end 2 of the protective sleeve 1, and the various sectional planes D, E and F are shown here and are also shown in details in FIGS. 5, 6 and 7.

FIG. 5 shows a cross section of the protective sleeve 1 along the line D-D in FIG. 4. The cross section shown here is in principle the same over the entire length of the protective sleeve 1 one except for the area in which openings are provided. The protective sleeve 1 is on the whole a single tube which is provided with a tube closure 7 on the proximal end. This tube closure may be a glued-in-place spigot or may just be a welded seam and/or a glued-on cover.

The tube 4 has an outer lumen 40, which is formed by an outer wall 41, preferably cylindrical. An inner lumen 42 is provided eccentrically inside the outer lumen 40. This inner lumen 42 is formed by an inner wall 43, preferably cylindrical. The terms “inner lumen 42” and “outer lumen 40” are in principle obtained due to the fact that in principle a smaller circle is shown geometrically in another circle, such that the two circles run so that the smaller circle practically does not touch the larger circle at any point. This design results in a sickle-shaped outer lumen 40 and a lumen 42 having a circular cross section in the preferred embodiment.

The cylindrical walls 41 and 43 of the two lumens 40 and 42 thus have a shared wall segment 44. If the inner lumen 42 is situated further toward the outside eccentrically, the shared wall segment 44 will be thinner. The central longitudinal axis 45 of the outer wall 41 and the central longitudinal axis 46 of the inner wall 43 define a plane that intersects the shared wall segment 44 along a line 47, forming the thinnest location in the area of the shared wall segment 44. The wall thickness of the shared wall segment 44 is so thin, at least in the area close to the line 47, that a membrane 49 is formed. This area is labeled as 48 in the figure.

The inner lumen 42 now serves to introduce a reusable measuring probe catheter 6. The same lumen may of course also be used for inserting a guide wire. If the protective sleeve 1 is in the correct position, then the guide wire can be extracted, and a measuring probe catheter 6 can be inserted.

In certain cases, at least the area 48 of the membrane 49 is so thin that a pressure sensor beneath it will respond with sufficient accuracy. If this is not the case, then a pressure sensor window 11, as shown in FIG. 6, may be provided in the area of the shared wall segment 44. FIG. 6 shows a section through the tube 4 of the protective sleeve in the area of the pressure sensor window which is situated essentially above the area 48 of the thinnest location but is larger than this thinnest location. In order for the protective sleeve 1 to nevertheless have the function of a protective sleeve, a thin film 110 is applied over the pressure sensor window 11. This film 110 may be welded or glued to the outside of the tube 4 along the location 111 around the pressure sensor window 11 at the side. Since this welding or gluing is not without problems, instead of merely using one film piece 110, this film 110 is preferably sealed by a heat-shrink tube section 112. Such a heat-shrink film tube may have a wall thickness of only a few hundredths of a millimeter. Preferably such a heat-shrink film tube wall thickness will be selected to be in the range between two and ten hundredths of a millimeter. In the shrunken state, such a heat-shrink tube section 112 is absolutely tightly in contact with the tube 4 under tension and covers the pressure sensor window 11, forming an absolute seal. A force F acting on the film 110 and/or the heat-shrink tube 112 then acts practically directly on the pressure sensor beneath it with virtually no distortion. In this approach, a pressure which is acting practically unidirectionally in the area of the pressure sensor window 11 is thus detected. However, if one would like to measure the pressure multidirectionally, i.e., regardless of the direction of the force F and also regardless of where this pressure acts along the circumference of the tube 4, then it is also possible to apply a second heat-shrink film tube section 113 over the heat-shrink tube section 112 so that they correspond and to introduce a gel or a liquid 114 between them. The two heat-shrink film tube sections 112 and 113 which are positioned one above the other with the gel 114 arranged between them form a cuff 115 which is shown in FIG. 9. In this case, however, the two heat-shrink tube film sections will be glued along the outer edges 116. The second outer film tube section 113 need not necessarily be designed as a shrinkage film but may also be manufactured from such a material, but this outer second film tube section 113 is at any rate not shrunk. This approach is depicted in detail in FIG. 9. On the other hand, FIG. 8 shows the unidirectional approach which has been discussed already with reference to FIG. 6, shown here in a perspective view.

FIG. 7 again shows the cross section of the tube 4, cut in the plane according to lines F-F in FIG. 4. This section is located in the rinse opening area 8, where it is shown in the area of two rinse openings 9. If one again assumes a plane spanned by the central longitudinal axis 45 of the outer wall 41 and the central longitudinal axis 46 of the inner wall, then the two rinse openings 9 are arranged with a lateral symmetrical offset to this plane of symmetry. The plane of symmetry is labeled as S. Thanks to this arrangement of the rinse openings 9, a relatively large volume of rinsing fluid can be introduced per unit of time in a bladder, for example. In the case of a pressure sensor catheter used for urology, the pressure sensors and/or the pressure sensor windows 11 in the inserted state are in the area of a ureter, whereas the rinse openings are then inside the bladder. The abdominal musculature causes the abdominal cavity to contract when the patient coughs, thereby exerting a pressure on the bladder so that a surge of the rinsing solution is expressed and then detected by the pressure sensors. However, the pressure in the bladder can also be merely successively increased until the sphincter muscle can no longer withstand this pressure. These values can then be analyzed diagnostically. Although one measurement in the unidirectional direction may be sufficient for the processes just described, abnormalities in the urethra/ureter can be measured by means of a pressure sensor catheter designed according to the embodiments in FIG. 4 in a protective sleeve. Such abnormalities may include any type of changes in the cross section. As soon as the cuff 115 enters the area of such a change in cross section, in particular a constriction of the cross section, the pressure on the cuff is increased and the gel conducts this pressure into the area of the pressure sensor window 11, so that the pressure sensor can respond to it.

Although for urological applications, in most cases only two pressure sensor windows 11 which are mounted at a certain distance from one another are provided, the protective sleeve 1 according to the invention may also be used for gastrological examinations, but here the dimensions of the protective sleeve 1 are much larger. Furthermore, in a gastrological examination not only is the length much greater but also accordingly a great many more measuring sites with corresponding pressure sensors are used. For gastrological use, up to 36 pressure sensors with corresponding pressure sensor windows may also be easily mounted here. Since the inner lumen 42 may have a substantial diameter, the corresponding number of electric conductors can also be accommodated therein accordingly with no problem.

FIG. 10 also shows the possibility of attaching contact surfaces which are to be attached to the protective sleeve from the outside. These contact surfaces are used to detect the leakage of even minimal amounts of urine and/or rinse solution from the bladder into the ureter. These contact surfaces 120 can preferably be applied to a self-stick backing film 121 by means of a coating. The conductors can likewise be applied from the contact surface 120 to the backing film 121. These conductors 122 run from the contact surfaces 120 to the distal end of the protective sleeve, where they are picked up to be able to measure a corresponding throughput signal. An insulating and protective cover layer 123 is attached over the printed conductors 122 and to some extent over the edge area of the contact surfaces 120. This cover layer 123 may be, for example, a corresponding insulating coating. The self-stick backing film with the printed conductors 122 of the contact surfaces 120 applied to it and the cover layer 123 may be applied to the desired locations on the outside of the tube 4 after finishing the protective sleeve 1. Normally the contact surfaces 120 are attached at a slight distance distally from the pressure sensor window 11, which is farthest from the proximal end.

An especially preferred embodiment of the protective sleeve 1 is shown in FIGS. 11 and 12. The essential difference here is that the shared wall segment 44 here is designed as a flat surface. This has the advantage that the known inherent stiffness of a rounded surface is reduced in this area. With this embodiment a corresponding pressure sensor window 11 can also be attached more easily and in a more defined manner. Subsequent attachment of a cover film or a heat-shrink tube section over the area of the pressure sensor window 11 is not affected by this.

Furthermore, the thinnest area 48 is already easily designed to be so thin that even a pressure sensor window 11 may be unnecessary. Furthermore, this thinnest area 48 is much greater than with the embodiment of the protective sleeve 1 described previously thanks to the flat course over a significant distance than in the embodiment of the protective sleeve 1 described previously.

Although it is of course then also possible to form the inner lumen 42 through an inner cylindrical wall in the case of such a design with a planar section of the shared wall section, in the example shown here, the cross section of the inner lumen 42 is designed to be rectangular. This rectangular or square cross-sectional shape offers additional advantages. Accordingly, the pressure sensor, which can be inserted with its pressure-sensitive surface into the inner lumen 42, may likewise be designed so that it comes to lie precisely flatly over the shared wall segment 44 and thus under the thinnest area 48 of the protective sleeve 1. In such an embodiment, a mechanism by which the pressure sensor must be rotatable after being inserted is consequently unnecessary. The alignment of the pressure sensor with the pressure sensor window 11 and/or with the thinnest area 48 is automatically obtained with a correct insertion of the reusable pressure sensor. The operating personnel need only be sure to insert the pressure sensor in the correct position. For this purpose, the pressure sensor may be labeled in color accordingly. Other visual identifiers may of course also be applied for position determination. The rectangular and/or square cross section of the reusable pressure sensor with the corresponding lines also results in the fact that this has less tendency to kink. Consequently, the skilled person will prefer this latter embodiment of the protective sleeve.

### LIST OF REFERENCE NUMERALS

1 protective sleeve

2 proximal end of the protective sleeve

3 distal end of the protective sleeve

4 tube

5 insertion connection

6 catheter

7 tube closure

8 rinse opening area

9 rinse openings

10 pressure-measuring area

11 pressure sensor window

12 electric resistance sensor

13 printed conductor

40 outer lumen

41 outer cylindrical wall

42 inner lumen

43 inner cylindrical wall

43′ inner wall

44 shared wall segment

45 central longitudinal axis of the outer wall

46 central longitudinal axis of the inner wall

47 line

48 thinnest area

49 membrane

50 nipple

51 insertion opening

52 feeder line

53 lateral opening

54 operating lever

110 film

111 adhesive or welded location

112 shrink tube section

113 second shrink tube section

114 gel or liquid

115 cuff

116 outer edge of the cuff

120 contact surfaces

121 backing film

122 printed conductors

123 cover layer

1. A protective sleeve (1) for a catheter (6) for performing intracorporeal measurements, characterized in that the protective sleeve (1) is a tube (4) which has an outer and an inner lumen (40, 42), such that the inner lumen (42) is arranged eccentrically in the outer lumen (40), such that an outer shared wall segment (44) of the two lumens is designed to be so thin that it forms a membrane (49) and at least one inner lumen (42) is closed on the proximal end of the sleeve (2).

2. The protective sleeve according to claim 1, characterized in that both lumens (40, 42) are closed on the proximal end (2).

3. The protective sleeve according to claim 1, characterized in that the inner lumen (42) has a circular cross section and the outer lumen (40) has a sickle-shaped cross section.

4. The protective sleeve according to claim 1, characterized in that the outer shared wall segment serves as a contact membrane, which is in contact with one or more pressure sensors arranged in the inner lumen, and whose connecting lines are protected and lead to the distal end toward the outside through this lumen.

5. The protective sleeve according to claim 1, characterized in that the outer shared wall has openings in the area of which pressure sensors come to lie such that the openings are closed and sealed by a heat-shrink tube section, and the heat-shrink tube section forms a highly flexible membrane.

6. The protective sleeve according to claim 5, characterized in that a second ring-shaped film section is sealed at both ends over the heat-shrink tube section, so that a chamber is formed between the heat-shrink tube section and the ring-shaped film section.

7. The protective sleeve according to claim 6, characterized in that the chamber is filled with gas and/or a gas mixture.

8. The protective sleeve according to claim 6, characterized in that the chamber is filled with a liquid or a gel.

9. The protective sleeve according claim 1, characterized in that the outer lumen has a plurality of openings at least approximately opposite the outer shared wall.

10. The protective sleeve according to claim 1, characterized in that several openings through the protective sleeve are present in the outer lumen on both sides of a plane of symmetry.

11. The protective sleeve according to claim 1, characterized in that at least a pair of contact surfaces are applied to the outer surface of the protective sleeve, conductors leading from these contact surfaces to the distal end of the sleeve.

12. The protective sleeve according to claim 11, characterized in that the contact surfaces and the conductors are applied to a self-stick film and the conductors are implemented as printed conductors over which an insulating cover layer is applied.

13. The protective sleeve according to claim 12, characterized in that the two contact surfaces form two non-contact half rings of at least approximately the same size in the state in which they are applied to the protective sleeve.

14. The protective sleeve according to claim 1, characterized in that a coupling piece is connected to the distal end of the protective sleeve.

15. The protective sleeve according to claim 14, characterized in that the coupling piece has an insertion funnel for a reusable pressure sensor unit which is connected to the inner lumen and a connection is connected to the outer lumen.

16. The protective sleeve according to claim 1, characterized in that the shared wall segment (44) is designed as a flat surface.

17. The protective sleeve according to claim 16, characterized in that the inner lumen (42) has a rectangular or square cross section such that the width of the shared wall segment (44) is equal to the width of a rectangular side and/or a side of a square of the inner wall (43′) of the lumen (42).
